(19) United States
(12) Patent Application Publication
(10) Pub. No.: US 2026/0224355 A1
(43) Pub. Date: Aug. 6, 2026

(54) VALVE PROSTHESIS WITH REDUCED PACKING DENSITY

(71) Applicant: Medtronic, Inc., Minneapolis, MN (US)

(72) Inventor: Victoria T. TIEN, Lakeville, MN (US)

(21) Appl. No.: 19/147,887

(22) PCT Filed: Jan. 18, 2024
(86) PCT No.: PCT/IB2024/050492

§ 371 Date: Jul. 14, 2025

Related U.S. Application Data
(60) Provisional application No. 63/481,816, filed on Jan. 27, 2023.

(51) Int. Cl. A61F 2/24; A61F 2/848

(52) U.S. Cl. CPC A61F 2/2418; A61F 2002/8483; A61F 2210/0076; A61F 2230/0067; A61F 2250/0039

(57) ABSTRACT

A transcatheter heart valve prosthesis includes an inner frame, an outer frame, and prosthetic valve coupled to the inner frame, an inner skirt coupled to the inner frame, and an outer skirt coupled to the outer frame. The inner frame includes an inflow end and an outflow end. The inner skirt extends from the inflow end of the inner frame and terminals proximal of the outflow end of the inner frame. The outer frame includes a first end and a second end. The first end of the outer frame is coupled to the inner frame between the inflow and outflow ends of the inner frame. The outer skirt extends from the first end of the outer frame and terminates proximal of the second end of the outer frame.

### CROSS-REFERENCE TO RELATED APPLICATION

This application claims the benefit of U.S. Provisional Patent Application Ser. No. 63/481,816, filed Jan. 27, 2023, the entire content of which is incorporated herein by reference.

### FIELD OF THE INVENTION

The present invention relates to heart valve prostheses, and more particularly, valve prostheses having a low profile.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems, and are delivered in a radially compressed or crimped configuration for advancement through the patient's vasculature. Accordingly, once positioned at a treatment site, a heart valve prosthesis may be expanded, or permitted to return to an uncompressed state, to engage tissue at the diseased heart valve region to, for instance, hold the heart valve prosthesis in position.

While these valve prostheses offer minimally invasive methods for heart valve repair and/or replacement, challenges remain such as reducing the profile of a heart valve prosthesis while maintaining required performance in vivo. The profile of a heart valve prosthesis refers to its radial dimension (e.g. diameter) when radially compressed for delivery. One challenge that relates to providing a mitral valve prostheses with a lower profile is minimizing the unintended movement of blood between the atrium and the ventricle, otherwise known as regurgitation. Current solutions use a combination of skirts positioned about a heart valve prosthesis. However, many current skirt configurations may be too thick/large so as to not permit a suitable reduction in profile for a mitral valve prosthesis. Therefore, there exists a need for improved skirt configurations in order to reduce an overall profile size of a mitral valve prosthesis while maintaining proper sealing thereabout when implanted at a target location.

The present disclosure relates to improvements in a heart valve prosthesis that ensures the heart valve prosthesis has a low profile for transcatheter delivery through a patient's vasculature, prevents left ventricular outflow tract (LVOT) obstruction, and increases conformity to the mitral annulus.

### BRIEF SUMMARY OF THE INVENTION

In accordance with first example hereof, a transcatheter heart valve prosthesis includes an inner frame extending from an inner frame first end to an inner frame second end, an outer frame extending from an outer frame first end and an outer frame second end, the outer frame first end being coupled to the inner frame between the inner frame first end and the inner frame second end, and the outer frame second end extending towards the inner frame second end. The transcatheter heart valve prosthesis further includes a prosthetic valve having a plurality of leaflets, the prosthetic valve coupled to the inner frame at a location between the inner frame first end and the inner frame second end, an inner skirt coupled to the inner frame, and an outer skirt coupled to outer frame. The inner skirt extends from the inner frame first end and terminates between the inner frame first end and the inner frame second. The outer skirt extends from the outer frame first end and terminates between the outer frame first end and the outer frame second end.

In a second example, in the transcatheter heart valve prosthesis according to any of the previous or subsequent examples herein, the plurality of leaflets of the prosthetic valve are coupled to the inner skirt along a joint line.

In a third example, in the transcatheter heart valve prosthesis according to any of the previous or subsequent examples herein, the plurality of leaflets and the inner skirt are coupled to the inner frame at the joint line.

In a fourth example, in the transcatheter heart valve prosthesis according to any of the previous or subsequent examples herein, the joint line curves toward and away from the inner frame first end.

In a fifth example, in the transcatheter heart valve prosthesis according to any of the previous or subsequent examples herein, the inner skirt terminates at a proximal end of the leaflets of the prosthetic valve.

In a sixth example, in the transcatheter heart valve prosthesis according to any of the previous or subsequent examples herein, the outer frame first end is coupled to the inner frame at the joint line.

In a seventh example, in the transcatheter heart valve prosthesis according to any of the previous or subsequent examples herein, the outer frame first end is coupled to the inner frame at a proximal-most end of the joint line.

In an eighth example, in the transcatheter heart valve prosthesis according to any of the previous or subsequent examples herein, a proximal end of the outer skirt is disposed at a distal end of the inner skirt or proximal of the distal end of the inner skirt.

In a ninth example, in the transcatheter heart valve prosthesis according to any of the previous or subsequent examples herein, the outer skirt terminates along the outer frame at a location longitudinally aligned with the inner frame second end.

In a tenth example, in the transcatheter heart valve prosthesis according to any of the previous or subsequent examples herein, the outer frame is shape-set with curvatures configured to conform to a native mitral annulus.

In an eleventh example, in the transcatheter heart valve prosthesis according to any of the previous or subsequent examples herein, the outer frame further includes barbs extending radially outward configured to prevent migration of the transcatheter heart valve prosthesis.

In a twelfth example, in the transcatheter heart valve prosthesis according to any of the previous or subsequent examples herein, the inner frame from the joint line to the inner skirt second end does not include a skirt.

In a thirteenth example, in the transcatheter heart valve prosthesis according to any of the previous or subsequent examples herein, the outer frame second end is uncovered.

In a fourteenth example, in the transcatheter heart valve prosthesis according to any of the previous or subsequent examples herein, the inner skirt is coupled to an interior surface of the inner frame.

In a fifteenth example, in the transcatheter heart valve prosthesis according to any of the previous or subsequent examples herein, the inner skirt is coupled to an exterior surface of the inner frame.

In a sixteenth example, in the transcatheter heart valve prosthesis according to any of the previous or subsequent examples herein, the outer skirt is coupled to an interior surface of the outer frame.

In a seventeenth example, in the transcatheter heart valve prosthesis according to any of the previous or subsequent examples herein, the outer skirt is coupled to an exterior surface of the outer frame.

In an eighteenth example, in the transcatheter heart valve prosthesis according to any of the previous or subsequent examples herein, the inner skirt comprises a fabric or other flexible and biocompatible material, or bovine or porcine pericardium tissue.

In a nineteenth example, in the transcatheter heart valve prosthesis according to any of the previous or subsequent examples herein, the outer skirt comprises a fabric or other flexible and biocompatible material, or bovine or porcine pericardium.

In a twentieth example, in the transcatheter heart valve prosthesis according to any of the previous or subsequent examples herein, the inner frame first end is an inflow end and the inner frame second end is an outflow end.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIGS. 1 and 2 are schematic illustrations of a mammalian heart having native heart valve structures.

FIG. 3 shows a side view of a heart valve prosthesis according to embodiments hereof.

FIG. 4A shows an inner frame of the heart valve prosthesis of FIG. 3 according to embodiments hereof.

FIG. 4B shows an outer frame of the heart valve prosthesis of FIG. 3 according to embodiments hereof.

FIG. 4C shows the outer frame of FIG. 4B coupled to the inner frame of FIG. 4A according to embodiments hereof.

FIG. 5 shows the heart valve prosthesis of FIG. 3 deployed within a native mitral valve.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). Further, the following detailed description is merely exemplary in nature and is not intended to limit the invention or the application and uses of the invention. Although the description of embodiments hereof is in the context of the treatment of heart valves such as the pulmonary, aortic, mitral, or tricuspid valve, the invention may also be used in other body passageways where it is deemed useful. Furthermore, there is no intention to be bound by any expressed or implied theory presented in the preceding technical field, background, brief summary or the following detailed description.

Specific embodiments of the present invention are now described with reference to the figures. The terms “inflow” and “outflow”, when used in the following description refer to a native vessel, native valve, or a device to be implanted into a native vessel or native valve, such as a heart valve prosthesis, are with reference to the direction of blood flow. Thus, “inflow” refers to positions in an upstream direction with respect to the direction of blood flow and the term “outflow” refers to positions in a downstream direction with respect to the direction of blood flow.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

Further, numerical terms such as “first,” “second,” “third,” etc. used herein are not meant to be limiting such that use of the term “second” when referring to a part in the specification does not mean that there necessarily is a “first” of part in order to fall within the scope of the invention. Instead, such numbers are merely describing that the particular embodiment being described has a “first” part and a “second” part. The invention is instead defined by the claims, in which one or more of the numbered parts may be claimed.

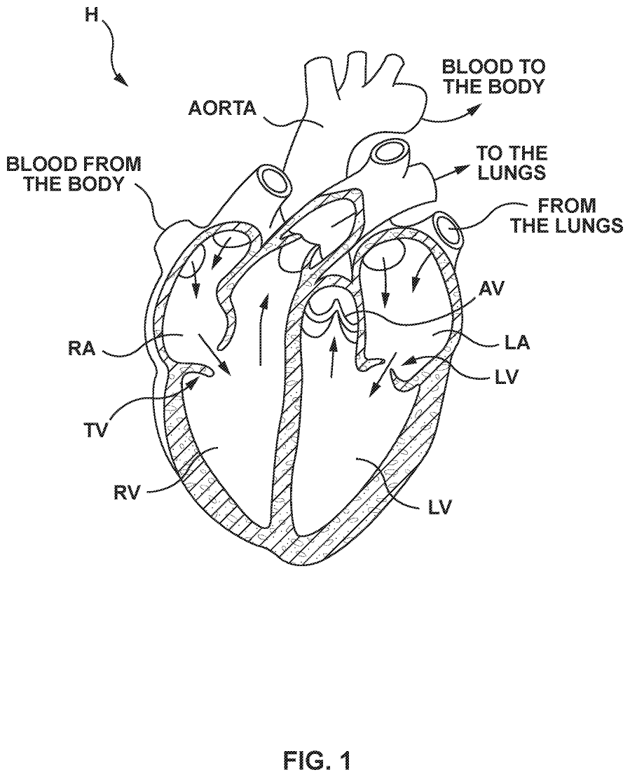

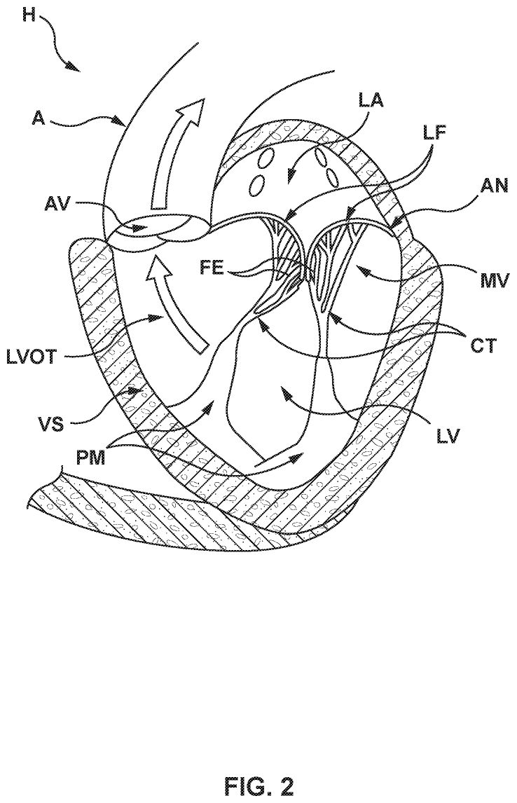

FIGS. 1 and 2 show an exemplary heart H. The heart H comprises a right atrium RA that receives deoxygenated blood from the body and pumps the deoxygenated blood through the tricuspid valve TV and into the right ventricle RV. The heart H also comprises a left atrium LA that receives oxygenated blood from the lungs via the pulmonary veins and pumps the oxygenated blood through the mitral valve MV into the left ventricle LV. The left ventricle LV contracts to redirect the blood flow outwardly through the aortic valve AV in the direction of the arrows. Back flow of blood or “regurgitation” through the mitral valve MV is prevented since the mitral valve is configured as a “check valve” which prevents back flow when pressure in the left ventricle LV is higher than that in the left atrium LA.

As shown in FIG. 2, the mitral valve MV comprises a pair of leaflets having free edges FE which meet evenly, or “coapt” to close in between the left atrium LA and the left ventricle LV. The left ventricular outflow tract (LVOT) is a region of the left ventricle that lies between the mitral valve MV and the ventricular septum VS, as shown in FIG. 2. The aortic valve AV, disposed adjacent to the LVOT, allows the oxygen-rich blood to pass from the left ventricle LV into the aorta A.

Systems, devices and methods are provided herein for percutaneous implantation of prosthetic heart valves in a heart of a patient. In some embodiments, methods and devices are presented for the treatment of valve disease by minimally invasive implantation of artificial replacement heart valves. In one embodiment, the artificial replacement valve can be a prosthetic valve device suitable for implantation and replacement of a mitral valve between the left atrium and left ventricle in the heart of a patient. In another embodiment, the prosthetic valve device can be suitable for implantation and replacement of another valve (e.g., a bicuspid or tricuspid valve) in the heart of the patient. However, this is not meant to be limiting, and the heart valve prostheses described herein can be used at other native heart valves, for example, the native tricuspid valve, the native aortic valve, and the native pulmonary valve.

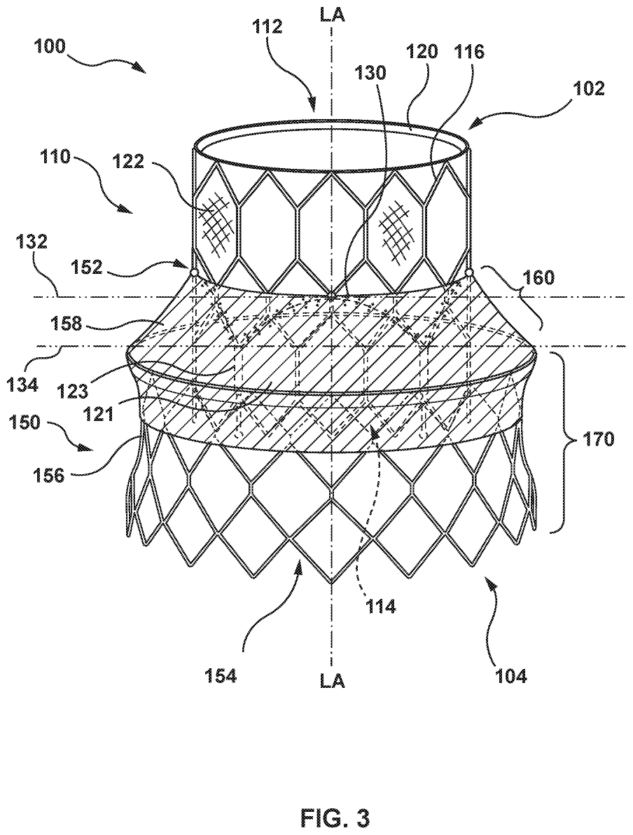

FIG. 3 shows a side view of a heart valve prosthesis 100 according to embodiments hereof. The heart valve prosthesis 100 includes an inflow end 102 and an outflow end 104. The heart valve prosthesis 100 further includes an inner frame 110 and an outer frame 150 that surrounds a portion of the inner frame 110. The inner frame 110 is configured to hold a prosthetic valve 120 in place and the outer frame 150 is configured to secure the heart valve prosthesis 100 to the native tissue of the heart when implanted in vivo. The heart valve prosthesis 100 may be considered to have a dual-stent structure, i.e., an inner stent and an outer stent. The inner frame 110 and outer frame 150 of the heart valve prosthesis 100 may be separately formed and then assembled together, or may alternatively be formed integrally as a single piece in the configuration shown in FIG. 3.

The inner frame 110 of the heart valve prosthesis 100, as shown in FIG. 3, is an expandable stent-like frame comprised of a plurality of thin interconnecting members referred to herein as struts 116, arranged in a variety of geometrical patterns. The inner frame 110 further includes an inflow end 112, an outflow end 114 that opposes the inflow end 112. The heart valve prosthesis 100 further comprises an inner skirt 122 coupled to the inner frame and to the prosthetic valve 120, as described in more detail below.

The outer frame 150 of the heart valve prosthesis 100, as shown in FIG. 3, is an expandable stent-like frame comprised of a plurality of struts 156, arranged in a variety of geometrical patterns. The outer frame 150 includes a first end 152 that is coupled to the inner frame 110 and a second end 154 that opposes the first end 152. An outer skirt 152 is coupled to the outer frame 152 and extends distally towards the second 154 from the first end 152 of the outer frame 150. The first end 152 of the outer frame 150 is coupled to the inner frame 110 at a location between the inflow end 112 and the outflow end 113 of the inner frame 110, which will be discussed in further detail below. The second end 154 of the outer frame 150 extends in a direction towards the outflow end 114 of the inner frame 110. The outer frame 150 comprises a connecting structure 160 extending from the first end 152 of the outer frame 150, and a fixation ring 170 that extends from the connecting structure 160 to the second end 154 of the outer frame 150, which will be described in further detail below.

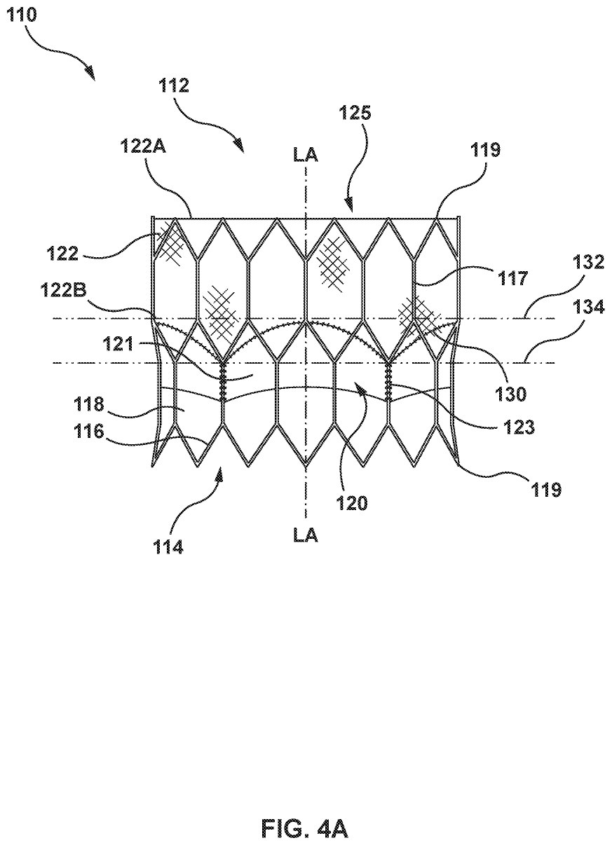

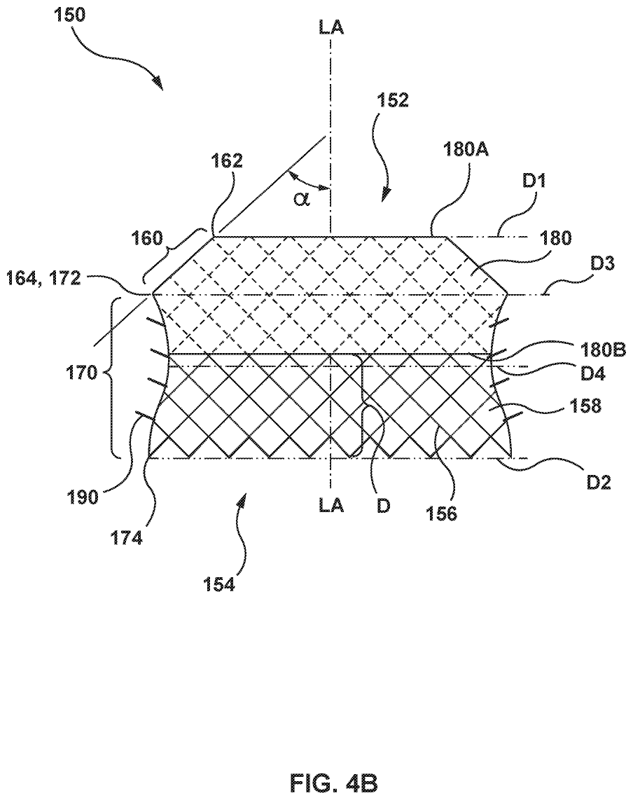

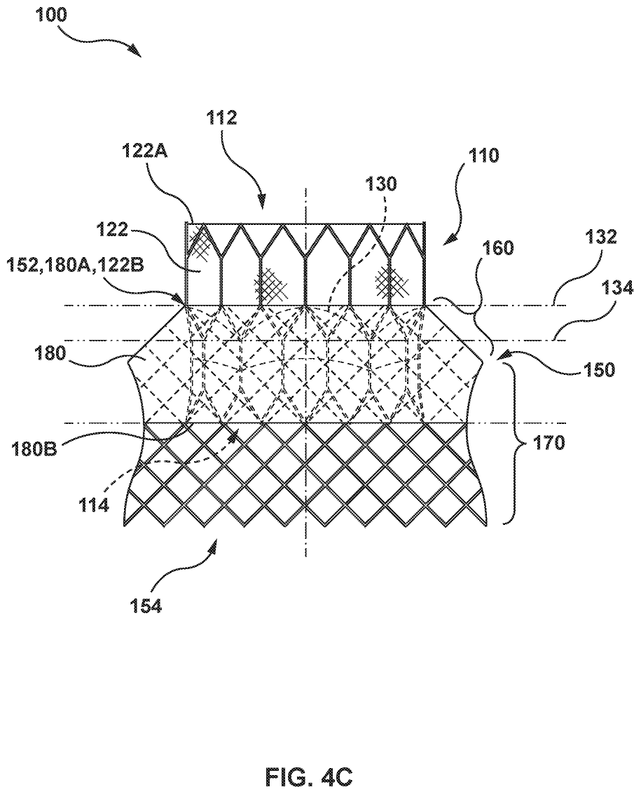

FIG. 4A shows the inner frame 110 of the heart valve prosthesis 100 without the outer frame 150 attached, for clarity purposes only. The inner frame 110 generally forms a hollow cylindrical shape around a central longitudinal axis LA, having a substantially constant diameter from the inflow end 112 to the outflow end 114 thereof. In the embodiment shown, the inner frame 110 is formed of angled struts 116 connected by crowns 119 forming cylindrical rows, and longitudinal struts 117 extending between the rows. However, this is not meant to be limiting, and any arrangement of struts in a stent-like fashion may be utilized. The struts 116, 117 and crowns 119 of the inner frame 110 define a plurality of open cells 118 arranged in a fence-like or honeycomb pattern. The inner frame 110 can have a longitudinal length, extending from the inflow end 112 to the outflow end 114, of about 20 mm-30 mm. The diameter of the inner frame 110 can be about 25 mm-29 mm.

The prosthetic valve 120 includes a plurality of leaflets 121 coupled to each other at lateral ends thereof at commissures 123, as shown in FIGS. 3 and 4A. In the embodiment shown, the prosthetic valve 120 includes three leaflets 121, but this is not meant to be limiting. The prosthetic valve 120 is configured to regulate flow through the inner frame 110 via the valve leaflets 121. When deployed in situ, the prosthetic valve 120 in a closed state is configured to block blood flow in one direction to regulate blood flow through the lumen 125 of the inner frame 110. The valve leaflets 121 are configured coapt with each other to close and are configured to open during diastole. The orientation of the leaflets 121 within the inner frame 110 depends upon which end of the heart valve prosthesis 100 is the inflow end 102 and which end of the heart valve prosthesis 100 is the outflow end 103, thereby ensuring one-way flow of blood through the heart valve prosthesis 100. The leaflets 121 are attached along their bases to the inner skirt 122, for example, using sutures or a suitable biocompatible adhesive, as described below.

The inner skirt 122 is coupled to the inner frame 110 and to the prosthetic valve 120. In the embodiment shown, the inner skirt 122 is coupled to an interior surface of the inner frame 110, but may instead be coupled to an exterior surface of the inner frame 110. The inner skirt 122 includes a first longitudinal end 122A and a second longitudinal end 122B. In an aspect hereof, the inner skirt 122 extends from the inflow end 112 of the inner frame 110 to a location between the inflow end 112 and the outflow end 114 of the inner frame 110. In other words, the first longitudinal end 122A of the inner skirt 122 is coupled to the inflow end 112 of the inner frame 110 and the second longitudinal end 122B of the inner skirt 122 extends in a direction towards the outflow end 114 of the inner frame 110, terminating about half way between the inflow end 112 and the outflow end 114 of the inner frame 110. In other words, the inner skirt 122 does not extend over the entire longitudinal length of the inner frame 110, i.e., the inner skirt 122 does not extend from the inflow end 112 to the outflow end 114, as shown in FIG. 4A. The inner skirt may comprise a fabric or other flexible and biocompatible material such as Dacron®, polytetrafluoroethylene (PTFE) expanded polytetrafluoroethylene (ePTFE), bovine or porcine pericardium, a polymer, thermoplastic polymer, polyester, stretched polytetrafluoroethylene (Gore-Tex®), a synthetic fiber, a natural fiber, and/or polyethylene terephthalate (PET), and/or any other suitable biocompatible material(s) or combinations of the above or other biocompatible materials.

When the heart valve prosthesis 100 is implanted within a native mitral valve, the inner skirt 122 is configured to limit the amount of unintentional blood leakage, otherwise known as regurgitation, between the left atrium LA and the left ventricle LV. The inner skirt 122 covers the interior surface of the inner frame 110 as to only allow blood to flow from the atrium to the ventricle when the heart valve prosthesis 100 is in an open state. The inner skirt 122 may take the form of a single piece or multiple pieces of material that is wrapped within the interior surface as to create a cylindrical body that is flush with the interior surface. The inner skirt 122 may be affixed to the inner frame 110 using sutures or adhesives, or may be encapsulated or dip-coated to the inner frame 110. In order to inhibit blood flow, the inner skirt 122 is further configured to substantially cover the cells 118 of the inner frame 110 disposed between the first end 122A and the second end 122B of the inner skirt 122.

The prosthetic valve 120 is coupled to the inner frame 110 at a location between the inflow end 112 and the outflow end 114 of the inner frame 110. As noted above, the base of each leaflet 121 is coupled to the skirt 122 along a curved joint line 130. The joint line 130 is also coupled to the inner frame 110, as shown in FIG. 4A. The base of each leaflet 121 may substantially semi-circle shaped, but this is not meant to be limiting. In the embodiment shown, the base of each leaflet 121 is also coupled to the inner skirt 122 at the joint line 130. Thus, in the embodiment shown, the leaflets 121, the inner skirt 122, and the inner frame 110 are coupled together along the joint line 130. However, this is not meant to be limiting. In other embodiments, for example, the inner skirt 122 may be coupled to the inner frame 111, and the leaflets 121 may be coupled to the inner skirt 122 without being directly attached to the inner frame 110. The curved joint line 130 may follow the contour of the cell pattern of the inner frame 110 so that most of the length of the joint line 130 is directly supported by the inner frame 110, thereby transmitting forces applied to the prosthetic valve 120 directly to the inner frame 110. However, this is not meant to be limiting, and the joint line 130 coupling the prosthetic valve 120 to the inner skirt 122 may instead be unsupported by the inner frame 110, or partially supported by the inner frame 110, as would be understood by those skilled in the art.

The second end 122B of the inner skirt 122 terminates at the bases of the leaflets 121. Accordingly, in the embodiment shown, the second end 122B of the inner skirt 122 terminates at the joint line 130 and is attached to the inner frame 110 at the joint line 130. As such, the inner frame 110 of the heart valve prosthesis 100 is uncovered, or open, from the joint line 130 to the outflow end 114 of the inner frame 110, as shown in FIG. 4A. Stated another way, the inner skirt 122 does not extend over the cells 118 of the inner frame 110 that are disposed between the joint line 130 and the outflow end 114 of the inner frame 110. The cells 118 of the inner frame 110 that are located between the joint line 130 and the outflow end 114 of the inner frame 110 are open and uncovered. As would be understood by those skilled in the art, the leaflets 121 may be located distal of the joint line 130. However, the leaflets 121 open and close, and as such do not cover the cells 118 between the joint line 130 and the outflow end 114 of the inner frame 110.

In the embodiment shown, the joint line 130 is curved. Therefore, the joint line 130 includes a proximal-most end 132 and a distal-most end 134, as shown in FIG. 4A. The proximal-most end 132 of the joint line 130 is defined by proximal-most points of each leaflet 121, i.e., the points on the curved joint line 130 that are disposed closest to the inflow end 112 of the inner frame 130. In the embodiment shown, the points are the peaks of the semi-circular shaped curves of the bases of the leaflets. In FIG. 4A, the proximal-most end of the joint line is shown as a line that intersects the proximal most points of the joint line. Similarly, the distal-most end 134 of the joint line 130 is defined by the distal-most points of the joint line, and in FIG. 4A, the distal-most end of the joint line is shown as a line intersecting the distal-most points of the joint line. In three-dimensions, the lines defining the proximal-most end 132 and the distal-most end 134 of the joint line are circumferential lines. In embodiments, the distance between the inflow-most end 132 and the outflow-most end 134 of the joint line 130 can be about 10 mm-20 mm. Accordingly, the inflow-most end 132 of the joint line 130 may be about 5 mm-15 mm from the inflow end 112 of the inner frame 110.

The leaflets 121 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject failure due to stretching, deforming, or fatigue.

FIG. 4B shows the outer frame 150 of the heart valve prosthesis 100 without the inner frame 110 attached, for clarity purposes only. The outer frame 150 is configured to secure the heart valve prosthesis 100 to the native valve and the surrounding tissue, such as the inward facing-surface of the native leaflets. The outer frame 150 generally forms a hollow cylindrical shape around the central longitudinal axis LA. The struts 156 of the outer frame 150 define a plurality of open cells 158 arranged in a fence-like or honeycomb pattern. The first end 152 of the outer frame 150 defines a first diameter D1 of the outer frame 150 and the second end 154 of the outer frame 150 defines a second diameter D2 of the outer frame 150 that is larger than the first diameter DI at the first end 152 when in a radially expanded configuration. The outer frame 150 may have a longitudinal length, extending from the first end 152 to the second end 154, of about 20 mm-30 mm. The first diameter D1 of the outer frame 150 in the radially expanded configuration may be about 25 mm-29 mm and the second diameter D2 of the outer frame 150 in the radially expanded configuration may be about 40 mm-55 mm.

As previously stated, the outer frame 150 of the heart valve prosthesis 100 includes the connecting structure 160 and the fixation ring 170. The connecting structure 160 is a portion of the outer frame 150 that is substantially conically-shaped and includes a first end 162 and a second end 164. The first end 162 of the connecting structure 160 begins at the first end 152 of the outer frame 150. The connecting structure 160 extends radially outwardly and in a downstream direction for the first end 162 to the second end 164 of the connecting structure 160. The connecting structure 160 extends radially outward and in a downstream directed at an angle a relative to the central longitudinal axis LA such that the first diameter D1 of the first end 162 is smaller than a third diameter D3 of the second end 164 of the connecting structure 160. The third diameter D3 of the outer frame 150 in the radially expanded configuration may be about 40-55 mm. The connecting structure 160 may be substantially straight from the first end 162 to the second end 164, as shown in FIG. 4B, or it may be curved. The angle a may be in the range of about 30°-45° relative to the central longitudinal axis LA. The connecting structure 160 can have a length, extending from the first end 162 to the second end 164, of about 5 mm-10 mm or a length equivalent to the width of 2 to 3 cells 158 of the outer frame 150, as shown in FIG. 4B.

The fixation ring 170 of the outer frame 150 includes a first end 172 and a second end 174. The first end 172 of the fixation ring 170 is integrally formed with the second end 164 of the connecting structure 160 of the outer frame 150. Thus, the first end 172 of the fixation ring 170 and the second end 164 of the connecting structure 160 define the third diameter D3 of the outer frame 150. The connecting structure 160 is configured to connect the fixation ring 170 to the inner frame 110. The second end 174 of the fixation ring 170 defines the second end 154 of the outer frame 150, as shown in FIG. 4B. In the embodiment shown, the fixation ring 170 is shape set with curvatures between the first end 172 and the second end 174 of the fixation ring 170 to allow for increased conformity to the native heart valve when implanted in vivo. However, this not meant to be limiting, and in other embodiments, the fixation ring may be substantially parallel to the central longitudinal axis LA and the fixation ring may conform to the anatomy by being relatively flexible. In some embodiments, the fixation ring 170 is shape set with curvatures that mirror or match the shape of a mitral annular to allow for improved conformity when deployed. As shown in FIG. 4B, in such an embodiment, the fixation ring 170 curves inward at a center portion of the fixation ring 170 such that the center portion defines a fourth diameter D4 of the outer frame 150 that is smaller than the second diameter D2 defined by the second end 174 of the fixation ring 170 and the third diameter D3 defined by the first end 172 of the fixation ring 170. The fourth diameter D4 of the outer frame 150 can be about 40 mm-50 mm. The outer frame 150 may further include one or more barbs 190 disposed on the exterior surface of the outer frame 150 to prevent migration of the heart valve prosthesis 100 once deployed in situ.

The outer frame 150 further includes an outer skirt 180. The outer skirt 180 may be coupled to an interior surface or an exterior surface of the outer frame 150, however, it is preferably disposed within and coupled to the interior surface of the outer frame 150. More particularly, the outer skirt 180 is coupled to the outer frame 150 such that the outer skirt 180 lines a portion of the interior surface of the outer frame 150, as shown in FIG. 4B. The outer skirt 180 includes a first end 180A and a second end 180B. In an aspect hereof, the outer skirt 180 extends from the first end 152 of the outer frame 150 to a portion between the first end 152 and the second end 154 of the outer frame 150. In other words, the first end 180A of the outer skirt 180 is coupled to the first end 152 of the outer frame 150 and the second end 180B of the outer skirt 180 terminates at about half the length of the outer frame. In other words, the outer skirt 180 does not extend over the entire longitudinal length of the outer frame 150, i.e., it does not extend from the first end 152 to the second end 154. Accordingly, a first distance D, measured from the second end 180B of the outer skirt 180 to the second end 154 of the outer frame 150, is left open and uncovered by the outer skirt 180, i.e., the outer skirt 180 terminates the first distance D proximal of the second end 154 of the outer frame 150. The cells 158 of the outer frame 150 that span the first distance D remain open and are not covered by the outer skirt 180, as shown in FIG. 4B. The first distance D can be about half the length of the outer frame 150. The outer skirt may comprise a fabric or other flexible and biocompatible material such as Dacron®, polytetrafluoroethylene (PTFE), expanded polytetrafluoroethylene (ePTFE), bovine or porcine pericardium, a polymer, thermoplastic polymer, polyester, stretched polytetrafluoroethylene (Gore-Tex®), a synthetic fiber, a natural fiber, and/or polyethylene terephthalate (PET), and/or any other suitable biocompatible material(s) or combinations of the above or other biocompatible materials.

FIG. 4C shows the assembled heart valve prosthesis 100 according to embodiments hereof. When assembled, the outer frame 150 of the heart valve prosthesis 100 is coupled to the inner frame 110 and extends radially outward from the inner frame 110 such that the outer frame 150 surrounds a portion of the inner frame 110. The inflow end 112 of the inner frame 110 defines the inflow end 102 of the heart valve prosthesis 100, as the inflow end 112 of the inner frame 110 is the proximal-most portion of the heart valve prosthesis 100. As can be seen, the inner skirt 122 extends from the inflow end 112 of the inner frame 110 to the joint line 130, where the inner skirt 122 is coupled to the bases of the leaflets 121 of the prosthetic valve 120. Further, the joint line 130 may be coupled to the inner frame 110, as discussed above.

As stated previously, the outer frame 150 is coupled to the inner frame 110 of the heart valve prosthesis 100. More particularly, the first end 152 of the outer frame 150 is attached to the inner frame 110 adjacent the joint line 130. More specifically, the first end 152 of the outer frame 150 is attached to the inner frame 110 at the proximal-most end 132 of the joint line 130, as shown in FIG. 4C. Accordingly, the first end 180A of the outer skirt 180 meets the second end 122B of the inner skirt 122 at the proximal-most end 132 of the joint line 130. The longitudinal continuity of the inner skirt 122 to the outer skirt 180 provides sealing and prevents or limits any unintentional blood leakage. In other words, from the proximal end 112 of the inner frame 110 to the second end 180B of the outer skirt 180, there is continuous skirt coverage of the inner frame 110 or the outer frame 150. It is preferable for the outer frame 150, and hence the first end 180A of the outer skirt 180, to be attached at the proximal-most end 132 of the joint line 130, rather than the distal-most end 134 of the joint line 130, so that there is no gap in coverage between the first end 180A of the outer skirt 180 and the second end 122B of the inner skirt 122 due to the curvature of the joint line 130. The first end 152 of the outer frame 150 can be attached to the inner frame 110 by suturing, riveting or welding, and/or other attachment mechanisms known to those skilled in the art.

The second end 154 of the outer frame 150 extends distally past the outflow end 114 of the inner frame 110. Stated another way, the outflow end 114 of the inner frame 110 terminates prior to the second end 154 of the outer frame 150. Accordingly, the second end 154 of the outer frame 150 defines the outflow end 104 of the heart valve prosthesis 100, as the second end 154 of the outer frame 150 is the distal-most portion of the heart valve prosthesis 100. The second end 154 of the outer frame 150 may extend about 5 mm-15 mm distally past the outflow end 114 of the inner frame 110.

As explained above, the second end 180B of the outer skirt 180 extends towards the second end 154 of the outer frame 150, but terminates proximally of the second end 154 of the outer frame 150. In the embodiment shown, the outer skirt 180 terminates at a location that is longitudinally aligned with the outflow end 114 of the inner frame 110, as shown in FIG. 4C. Stated another way, the second end 180B of the outer skirt 180 and the outflow end 114 of the inner frame 110 are longitudinally aligned. Thus, the cells 158 of the outer frame 150 that are disposed between the second end 180B of the outer skirt 180 and the second end 154 of the outer frame 150 remain open and are uncovered, as shown in FIG. 4C. The uncovered cells 158 between the second end 180B of the outer skirt 180 and the send 154 of the outer frame 150 assist in preventing or minimizing left ventricular outflow tract (LVOT) obstruction, as explained in further detail below.

The heart valve prosthesis 100 is configured to be compressed into a reduced-diameter delivery configuration within a delivery catheter and to return to an expanded, deployed configuration when delivered or released from the delivery catheter within a native mitral valve. As stated previously, the inner skirt 122 does not cover the entire inner frame 110 and the outer skirt 180 does cover the entire outer frame 150. The reduced amount of fabric of the inner skirt 122 and the outer skirt 180 reduces the packing density and overall profile of the heart valve prosthesis 100 when it is compressed or crimped into the delivery configuration. The reduced profile of the heart valve prosthesis 100 simplifies access, navigation and deployment of the prosthesis 100, particularly in patients with smaller vasculature.

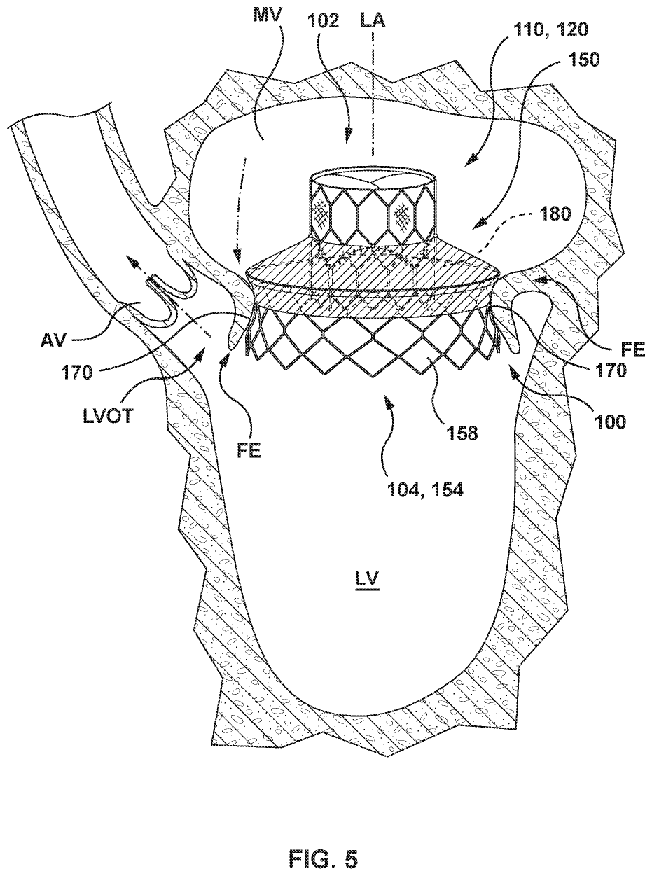

FIG. 5 shows the heart valve prosthesis 100 in an expanded or uncompressed configuration within a native mitral valve MV after it has been delivered to the mitral valve MV via a delivery catheter. As can be seen, the inflow end 102 of the heart valve prosthesis 100 is disposed partially within the left atrium LA and the outflow end 104 of the heart valve prosthesis 100 is disposed partially within the left ventricle LV. The heart valve prosthesis 100 is deployed between the free edges FE of the mitral valve MV leaflets such that the fixation ring 170 of the outer frame 150 contacts the free edges FE of the leaflets. The outer frame 150 of the heart valve prosthesis 100 secures the inner frame 110 and the prosthetic valve 120 disposed therein within the native mitral valve MV, thereby preventing migration or deformation of the inner frame 110 and the prosthetic valve 120 disposed therein, thereby reducing the risk of damage to the prosthetic valve 120. Blood flows from the left atrium LA, through the prosthetic valve 120 of the heart valve prosthesis 100, and more particularly through the leaflets 121 of the prosthetic valve 120 when the prosthetic valve 120 is open, into the left ventricle LV, and then through the LVOT and the aortic valve AV.

As mentioned previously, the left ventricular outflow tract (LVOT) is disposed adjacent to the mitral valve MV. A portion of the second end 154 of the outer frame 150 of the heart valve prosthesis 100 may partially protrude or extend into or around the LVOT. Because the second end 154 of the outer frame 150 is uncovered by the outer skirt 180, obstruction of blood flow through the LVOT is minimized or prevented. Blood is able to flow from the left ventricle LV, through the open cells 158 of the outer frame 150 to pass through the LVOT and the aortic valve AV, thus preventing LVOT obstruction.

It should be understood that the heart valve prosthesis shown and described herein can be used to replace a native mitral valve. However, this is not meant to be limiting, as one of ordinary skill in the art would understand that the heart valve prosthesis shown and described herein can also be configured for treating other valves of the heart such as the tricuspid valve. Many of the devices and methods disclosed herein can further provide for long-term (e.g., permanent) and reliable anchoring of the prosthetic device even in conditions where the heart or native valve may experience gradual enlargement or distortion. It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

1. A transcatheter heart valve prosthesis comprising:
an inner frame extending from an inner frame first end to an inner frame second end;
an outer frame extending from an outer frame first end to an outer frame second end,, the outer frame first end being coupled to the inner frame between the inner frame first end and the inner frame second end, and the outer frame second end extending towards the inner frame second end;
a prosthetic valve having a plurality of leaflets, the prosthetic valve coupled to the inner frame at a location between the inner frame first end and the inner frame second end;
an inner skirt coupled to the inner frame; and
an outer skirt coupled to the outer frame;
wherein the inner skirt extends from the inner frame first end and terminates between the inner frame first end and the inner frame second end, and
wherein the outer skirt extends from the outer frame first end and terminates between the outer frame first end and the outer frame second end.

2. The transcatheter heart valve prosthesis of claim 1, wherein the plurality of leaflets of the prosthetic valve are coupled to the inner skirt along a joint line.

3. The transcatheter heart valve prosthesis of claim 2, wherein the plurality of leaflets and the inner skirt are coupled to the inner frame at the joint line.

4. The transcatheter heart valve prosthesis of claim 2, wherein the joint line curves toward and away from the inner frame first end.

5. The transcatheter heart valve prosthesis of claim 1, wherein the inner skirt terminates at a proximal end of the leaflets of the prosthetic valve.

6. The transcatheter heart valve prosthesis of claim 2, wherein the outer frame first end is coupled to the inner frame at the joint line.

7. The transcatheter heart valve prosthesis of claim 6, wherein the outer frame first end is coupled to the inner frame at a proximal-most end of the joint line.

8. The transcatheter heart valve prosthesis of claim 1, wherein a proximal end of the outer skirt is disposed at a distal end of the inner skirt or proximal of the distal end of the inner skirt.

9. The transcatheter heart valve prosthesis of claim 1, wherein the outer skirt terminates along the outer frame at a location longitudinally aligned with the inner frame second end.

10. The transcatheter heart valve prosthesis of claim 1, wherein the outer frame is shape-set with curvatures configured to conform to a native mitral annulus.

11. The transcatheter heart valve prosthesis of claim 1, wherein the outer frame further includes barbs extending radially outward configured to prevent migration of the transcatheter heart valve prosthesis.

12. The transcatheter heart valve prosthesis of claim 2, wherein the inner frame from the joint line to the inner frame second end does not include a skirt.

13. The transcatheter heart valve prosthesis of claim 1, wherein the outer frame second end is uncovered.

14. The transcatheter heart valve prosthesis of claim 1, wherein the inner skirt is coupled to an interior surface of the inner frame.

15. The transcatheter heart valve prosthesis of claim 1, wherein the inner skirt is coupled to an exterior surface of the inner frame.

16. The transcatheter heart valve prosthesis of claim 1, wherein the outer skirt is coupled to an interior surface of the outer frame.

17. The transcatheter heart valve prosthesis of claim 1, wherein the outer skirt is coupled to an exterior surface of the outer frame.

18. The transcatheter heart valve prosthesis of claim 1, wherein the inner skirt comprises a fabric or other flexible and biocompatible material, or bovine or porcine pericardium tissue.

19. The transcatheter heart valve prosthesis of claim 1, wherein the outer skirt comprises a fabric or other flexible and biocompatible material, or bovine or porcine pericardium.

20. The transcatheter heart valve prosthesis of claim 1, wherein the inner frame first end is an inflow end and the inner frame second end is an outflow end.
